(19) United States
(12) Patent Application Publication
(10) Pub. No.: US 2024/0288447 A1
(43) Pub. Date: Aug. 29, 2024

(54) ANALYSIS OF VITREOUS SAMPLES TO GUIDE TREATMENT

(71) Applicant: Twenty Twenty Therapeutics LLC, South San Francisco, CA (US)

(72) Inventors: Alexandre R. Tumlinson, South San Francisco, CA (US); Dimitri Azar, South San Francisco, CA (US)

(21) Appl. No.: 18/113,150

(22) Filed: Feb. 23, 2023

(51) Int. Cl. G01N 33/68; C12Q 1/70; G01N 33/569

(52) U.S. Cl. CPC G01N 33/6893; C12Q 1/70; G01N 33/56911; G01N 2333/515; G01N 2333/5412; G01N 2333/5421; G01N 2469/10; G01N 2800/16; G01N 2800/52

(57) ABSTRACT

A method of guiding medical treatment of patients receiving an intravitreal injection are described. The method includes obtaining a vitreous sample from a subject receiving an intravitreal injection first treatment, determining the biomarker signature of the sample, reporting the biomarker signature of the sample, and changing the method of treatment to a second treatment if the biomarker signature indicates that use of the second treatment will have a preferable outcome.

### BACKGROUND

The vitreous humor is a colorless, gelatinous fluid within an eye or eyeball of an animal composed of approximately 98-99% water with trace amounts of hyaluronic acid, glucose, anions, cations, ions, and a fine network of collagen. Vitreous humor provides support to the surrounding structures of the eye, absorbs mechanical trauma, and provides circulation and regulation of oxygen, metabolites, and nutrients.

Pathology in the retina is driven by cellular and intracellular processes which include the transmission of signaling molecules which can be found in the vitreous compartment of the eye. A common route of modulating these processes is by injecting drugs into the vitreous compartment of the eye. Diagnosis of pathology and feedback on the effectiveness of intravitreally injected drugs is currently performed by imaging the retina through the anterior segment of the eye. This feedback loop currently takes many weeks to months, during which time, disease may be causing permanent damage. Similarly, the evaluation of side effects of treatment are largely measured by structural measurements. For example, inflammation of the vitreous may be monitored by looking for cellular infiltrates clouding the vitreous, or effects such as vasculitis that may occur with severe inflammatory side effects.

More effective diagnosis and feedback could be achieved if vitreous signaling molecules were sampled and analyzed and provided as inputs to the doctor's decision-making process. A first significant obstacle to more effective diagnosis is access to the vitreous sample. A second significant obstacle to chemical biomarker-based diagnosis is the need to develop a decision path that guides care when analysis is available

Disease states for most posterior eye disease and particularly neovascular disease are primarily evaluated by structural imaging. This view ignores the causes of structural changes and generally reports damage after it has happened. It is also not directly correlated to the pharmaceutical interventions used to treat the disease. Protein analysis of aqueous humor is easier to acquire and may correlate to vitreous proteomics changes over large populations. However, aqueous humor protein concentrations vary significantly from vitreous protein concentration significantly in any single subject however, and do not correlate well enough to retina conditions to predict appropriate therapy decisions.

### SUMMARY OF THE INVENTION

The present invention provides a method of guiding medical treatment. The method includes the steps of obtaining a vitreous sample from a subject receiving an intravitreal injection first treatment, determining the biomarker signature of the sample, reporting the biomarker signature of the sample, and changing the method of treatment to a second treatment if the biomarker signature indicate the second treatment will have a preferable outcome.

In some embodiments, the method includes simultaneously gathering vitreous samples by intravitreal injection (IVI) which are then analyzed for biomarkers. In additional embodiments, the sample is acquired during the routine administration of anti-VEGF drug, the sample is transferred frozen to an offsite laboratory, analyzed for signaling molecules involved in angiogenesis and inflammation pathways, and results returned to the doctor in less than one month. A result is then returned in time to meaningfully direct patient care. The care provider then uses the results to decide the appropriate interval for drug administration or the type of drug delivered in the next scheduled care session. For example, a test result with a high concentration of inflammatory components that are known to be weakly influenced by the currently administered drug argues toward the shift to a drug which more strongly modulates those inflammatory components. The results pathway may be enhanced by applying care decision logic to the analysis results and suggesting the care decision.

The first treatment will often have a specific spectrum of actions. For example, where the first treatment includes an IVI antiVEGF agent, it will act directly on a known set of proangiogenic molecules such as VEGF-165A, PIGF or others, to deactivate them, or the receptors the bind to. This binding may in turn have effects on other signaling molecules upregulating or downregulating them. The summation of all of these signaling molecules changes the disease state of the tissues (which may have further effects on the signaling environment). The analysis of the sample may determine the concentration of various biomarkers (e.g., proteins) in this signaling chain.

### BRIEF DESCRIPTION OF THE FIGURES

The present invention may be more readily understood by reference to the following figures, wherein:

FIG. 1 provides a schematic representation of the method of guiding medical treatment.

FIG. 2 provides a flow diagram showing the basic steps in the method of guiding medical treatment.

FIG. 3 provides a schematic illustration of an example combined biological sampling and injection assembly.

FIG. 4 provides a graph showing an embodiment of a doctor interface describing a few measured proteins and describing a history of treatments and results. All measured values are summarized in an angiogenic score which is used to track the tendency towards an event as the timing between treatments is optimized. Markers on the vertical axis indicate angiogenic score limits corresponding to maximum acceptable probability of angiogenic event and a highly unlikely probability of an angiogenic event.

FIG. 5 provides a bar graph indicating the concentration of multiple proteins in a sample. The doctor chooses between a first treatment that has been shown to reduce high elements on the right. Because the elements on the left are farther from normal, the doctor is guided to give the first treatment.

### DETAILED DESCRIPTION OF THE INVENTION

The present disclosure provides a method of guiding medical treatment of patients receiving an intravitreal injection. The method includes obtaining a vitreous sample from a subject receiving an intravitreal injection first treatment, determining the protein signature of the sample, reporting the protein signature of the sample, and changing the method of treatment to a second treatment if the protein signature indicates the second treatment will have a preferable outcome.

The terms “subject” and “patient” can be used interchangeably herein, and generally refer to a mammal, including, but not limited to, primates, including simians and humans, equines (e.g., horses), canines (e.g., dogs), felines, various domesticated livestock (e.g., ungulates, such as swine, pigs, goats, sheep, and the like), as well as domesticated pets and animals maintained in zoos. Treatment and evaluation of human subjects is of particular interest. Human subjects can be various ages, such as a child (under 18 years), adult (18 to 59 years) or elderly (60 years or older) human subject.

As used herein, the terms “treatment,” “treating,” and the like, refer to delivering care with an expected desirable pharmacologic or physiologic effect. The effect may be therapeutic in terms of a partial or complete cure for a disease or an adverse effect attributable to the disease. “Treatment,” as used herein, covers any treatment of a disease in a mammal, particularly in a human, and can include inhibiting the disease or condition, i.e., arresting its development; and relieving the disease, i.e., causing regression of the disease.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

A “biomarker” in the context of the present invention refers to a biological compound, such as a polynucleotide or polypeptide that may be found in a sample taken from a patient having an ophthalmic condition, and whose level may vary in response to an ophthalmic condition.

As used herein, a “biomarker signature” refers to a pattern of absolute or relative biomarker levels for one or more biomarkers that is characteristic of a particular disease or condition or stage of disease or condition.

“Prognosis” as used herein generally refers to a prediction of the probable course and outcome of a clinical condition or disease. A prognosis of a patient is usually made by evaluating factors or symptoms of a disease that are indicative of a favorable or unfavorable course or outcome of the disease. It is understood that the term “prognosis” does not necessarily refer to the ability to predict the course or outcome of a condition with 100% accuracy. Instead, the skilled artisan will understand that the term “prognosis” refers to an increased probability that a certain course or outcome will occur; that is, that a course or outcome is more likely to occur in a patient exhibiting a given condition, when compared to those individuals not exhibiting the condition.

“Nucleic acid” or “oligonucleotide” or “polynucleotide”, as used herein, may mean at least two nucleotides covalently linked together. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof. The term “nucleotide sequence,” as used herein, refers to an oligonucleotide, nucleotide, or polynucleotide of single-stranded or double stranded DNA or RNA, or fragments thereof.

Unless defined otherwise, all technical and scientific terms used herein have the same meaning as commonly understood by one of ordinary skill in the art to which this invention belongs.

As used herein and in the appended claims, the singular forms “a”, “and”, and “the” include plural referents unless the context clearly dictates otherwise. Thus, for example, reference to “a sample” also includes a plurality of such samples and reference to “the protein” includes reference to one or more protein, and so forth.

### Methods of Guiding Treatment

The present invention provides a method of guiding medical treatment. The method includes obtaining a vitreous sample from a subject receiving an intravitreal injection first treatment, determining the biomarker signature of the sample, reporting the biomarker signature of the sample, and changing the method of treatment to a second treatment if the biomarker signature indicates that use of the second treatment will have a preferable outcome. The method can be carried out a single time, or in some cases the biomarker signature can be determined and reported a plurality of times.

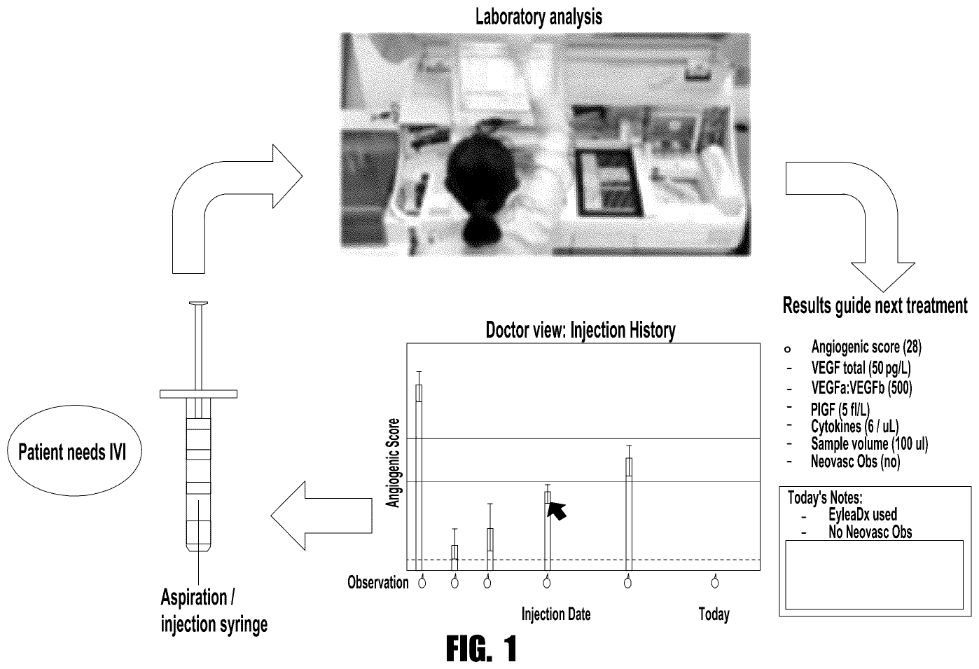

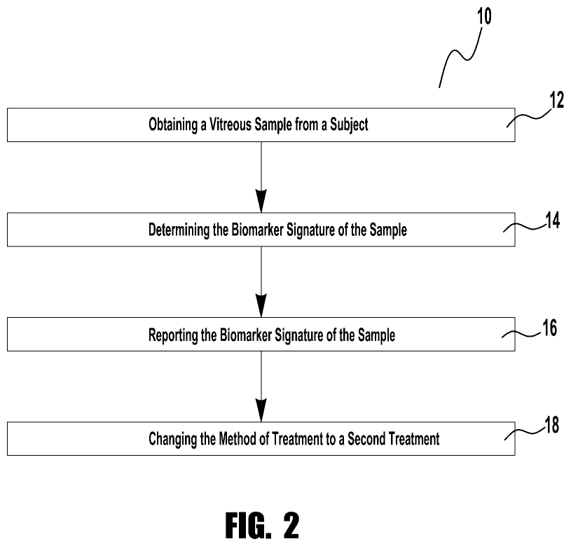

FIG. 1 provides a schematic representation of the method of guiding medical treatment. FIG. 2 provides a flow diagram showing the basic steps in the method of guiding medical treatment. The steps in the method of guiding treatment 10 include a) obtaining a vitreous sample from a subject receiving an intravitreal injection first treatment 12, b) determining the biomarker signature of the sample 14, c) reporting the biomarker signature of the sample 16, and d) changing the method of treatment to a second treatment if the biomarker signature indicates that use of the second treatment will have a preferable outcome 18.

The subject in the method is receiving an intravitreal injection first treatment. By “receiving” a treatment it is meant that the subject has received the treatment recently for treatment that is ongoing, or that the subject is administered the first treatment at the same time that vitreous sampling occurs. The specific agent being administered for the intravitreal injection (IVI) first treatment depends on the condition being treated. Examples of conditions where IVI is used include macular edema, macular degeneration, infection, uveitis, endophthalmitis, diabetic retinopathy, and retinal vein occlusion. For endophthalmitis the doctor will have a strong indication of the likely family of infectious organism, either bacterial, viral, or fungal.

In some embodiments, such as where the condition being treated is infection or endophthalmitis, the first IVI can be a broad-spectrum agent that is known to work against a variety of different infectious organisms. In some embodiments, the first treatment comprises administration of an anti-angiogenic agent suitable for ophthalmic conditions, while in other embodiments the first treatment comprises administration of an antibiotic. The specific agent being used can be determined based on the knowledge of one skilled in the art for treating ophthalmic conditions that can be treated using intravitreal injection. Examples of intravitreal medications include bevacizumab, ranibizumab, aflibercept, brolucizumab, faricimab, triamcinolone acetonide, ganciclovir, clindamycin, foscarnet, fomivirsen, methotrexate, vancomycin, ceftazidime, amikacin, amphotericin B, voriconazole, and dexamethasone.

The method includes the step of obtaining a vitreous sample from a subject. Vitreous sampling is a relatively common ophthalmic procedure. Methods of obtaining a vitreous sample include the use of a cutter or needle aspiration. A state-of-the-art miniature cutting tool may be delivered through a 23-gauge trocar. Needle aspiration may be performed with needles as small as 27-gauge. Vitreous samples are typically frozen or otherwise stabilized so that they can be processed in a laboratory outside of the hospital or ophthalmic office setting.

### Intravitreal Injection Devices

Intravitreal injection is also a common ophthalmic procedure. A variety of drugs can be delivered directly to the vitreous gel that supports the globe of the eye, such as anti-vascular endothelial growth factor (anti-VEGF) biologics and steroids. However, injection of medication into the vitreous or aqueous humor of the eye can increase the intraocular pressure by as much as 25 mmHg, which is substantially greater than the levels that are considered to be potentially harmful. Wu et al., Semin Ophthalmol., 24(2):100-5 (2009). Accordingly, in some embodiments, it is preferable to obtain the vitreous sample at the same time (i.e., simultaneous) that intravitreal injection is being carried out in order to avoid an increase in intraocular pressure. When conducting simultaneous injection and sampling, it can also be preferable to have the injection and sample volume being substantially the same. Note that similar principles could be applied to aqueous fluid acquired from the eye, which can be obtained instead or in addition to intravitreal sampling.

In some embodiments, the vitreous sample is obtained using a device that is configured to inject a volume into the eye having the same volume as the sample being obtained. See for example US Patent Publication No. 20210353455, entitled “Intravitreal Injection Device with Combined Vitreous Sampling,” or US Patent Publication No. 20220370293, entitled “Combined Biological Sampling and Injection Assemblies and Associated Devices, Systems, and Methods,” the disclosures of which are incorporated herein by reference. Use of such devices facilitates the simultaneous injection and sampling where the injection and sample volume are substantially the same.

As described by US Patent Publication No. 20210353455, in some embodiments, the device used to obtain the vitreous sample is a double-barreled syringe comprising an injectant chamber and a sample chamber. For example, the device can be an injection and extraction ophthalmic device comprising a housing configured to hold: an injectant chamber configured to store an injectant; a plunger disposed within the injectant chamber; and a stopper coupled to a distal portion of the plunger; a hypodermic needle disposed at a distal end of the housing; and a sample chamber located within the housing adjacent to the distal end, wherein the housing, hypodermic needle, and sample chamber are arranged such that: when the stopper is moved to a first depth within the housing, the sample chamber is configured to receive material via the hypodermic needle; and when the stopper is moved to a second depth within the housing exceeding the first depth, the injectant chamber is configured to dispense the injectant through the hypodermic needle. In some embodiments, the housing is a double-barreled syringe, and wherein the injectant chamber is disposed within a first barrel of the housing in fluid communication with the hypodermic needle, and the sample chamber is disposed within a second barrel of the housing in fluid communication with the hypodermic needle.

As described by US Patent Publication No. 20220370293, in some embodiments, the device used to obtain the vitreous sample is a sampling device for treating and diagnosing ophthalmic disorders that includes a container having a body defining a chamber and an opening to the chamber. A septum is bonded to the container to cover the opening and hermetically seal the chamber. The septum has an adherent layer with a first thickness and including a material configured to adhere to a rim of the container to provide a hermetic seal with the container. A metallic foil layer having a second thickness is coupled to the adherent layer. An elastomeric layer having a third thickness is coupled to the metallic foil layer to position the metallic foil layer between the adherent layer and the elastomeric layer. The third thickness is greater than the sum of the first thickness and the second thickness.

In some embodiments, a “pencil grip” vitreous aspiration and drug delivery device is provided. This is described by or U.S. Patent Application No. 63/417,798, entitled “Pencil Grip Vitreous Aspiration and Drug Delivery Device,” the disclosure of which is incorporated herein by reference. This device also allows for injection and withdrawal of comparable-sized samples from the vitreous humor. In one embodiment, the “pencil grip” device is a victrectomy device includes a body extending along an axis from a proximal end to a distal end and having a central passage. A hollow eye penetration member is provided for insertion into the vitreous humor of an eye and extends within the central passage and out of the distal end of the body. An assembly including a chamber under vacuum and a piercing member extend away from the chamber. The chamber is movable in a direction transverse to the centerline from a first position in which the chamber is not fluidly connected to the eye penetration member to a second position in which the piercing member fluidly connects the chamber to the eye penetration member for drawing a vitreous sample into the chamber under vacuum pressure.

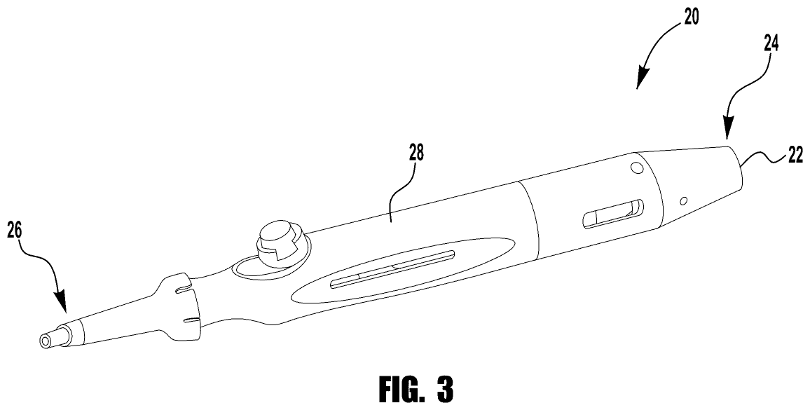

FIG. 3 provides an image of an embodiment of the “pencil grip” vitreous aspiration and drug delivery device. The device 20 has a pencil-grip design and extends along a centerline 22 from a first or proximal end 24 to a second or distal end 26, and having a central passage 28 extending generally along the centerline 22. The device 20 is operable by a user, e.g., a physician, to obtain a sample of vitreous humor from an eye of a patient.

### Biomarker Signature

The method also includes determining the biomarker signature of a sample. The biomarker signature can reflect the results of the determination of one, two, three, four, or five or more biomarkers. In some embodiments, the protein signature comprises a weighted sum of the concentrations of a plurality of proteins.

The biomarkers can be proteins, microscopic structure, surface antigens, or polynucleotides (e.g., RNA or DNA), and other biochemical factors such as vitamin or mineral levels. In some embodiments, the biomarker signature comprises a protein, and the biomarker signature can be referred to as a protein signature, while in other embodiments, the biomarker signature comprises a polynucleotide, and the biomarker signature can be referred to as a polynucleotide signature. In some embodiments, the protein signature consists of only protein biomarkers, and in some embodiments, the polynucleotide signature consists of only polynucleotide signatures. In some embodiments, the biomarker signature includes bacterial antigens, while in further embodiments the biomarker signature includes viral RNA. Analysis of the biomarkers for an infectious agent may confirm the specific type of infectious agent present and if the treatment given or an alternative is likely to be successful.

In some embodiments, the biomarker signature is a protein signature. In further embodiments, the protein signature includes angiogenic factors such as vascular endothelial growth factor (VEGF), placental growth factor (PIGF), or angiopoietin 2 (Ang2). In yet further embodiments, the protein signature comprises inflammatory factors such as interleukin-8 (IL-8) and/or interleukin-6 (IL-6). The biomarkers can be identified and quantified using analysis methods known to those skilled in the art.

In some embodiments, the protein signature comprises proteins that are the known targets of ophthalmic drugs. A variety of different proteins are known to be affected by ophthalmic drugs, as understood by those skilled in the art. Examples of proteins that are the known targets of ophthalmic drugs include angiopoietin-2 (Ang2), Integrin alpha 5 (ITGA5), Integrin beta (ITGB), interleukin-10 (IL10), recombinant interleukin-6 (IL6-R), interleukin-6 (IL6), interleukin-8 (IL8), kalikreins 4, 6, 8, 10, 11, 12, 13, and 14, monocyte chemoattractant protein-1 (MCP-1), placental growth factor (PGF), platelet derived growth factors PDGF-C, PDGF-Ra, PDGF-RB, PDGFA, and PDGFB, pleiotrophin (PTN), vascular endothelial growth factor A (VEGFA), vascular endothelial growth factor D (VEGFD), vascular endothelial growth factor 1 (VEGFR1), vascular endothelial growth factor receptor 2 (VEGFR2), vascular endothelial growth factor 3 (VEGFR3), connective tissue growth factor (CTGF), vascular endothelial growth factor B (VEGF-B), vascular endothelial growth factor C (VEGF-C), protein kinase C beta (PKC-B), integrin αvβ3, integrin α5β1, and bradykinin.

In some embodiments, the ophthalmic drugs are antibody-based drugs that specifically bind to the proteins. An antibody “specifically binds” when the antibody preferentially binds a target structure, or subunit thereof, but binds to a substantially lesser degree or does not significantly bind to a biological molecule that is not a target structure. The term antibody, as used herein and unless further limited, refers to single chain, two-chain, and multi-chain proteins and glycoproteins belonging to the classes of polyclonal, monoclonal, chimeric and hetero immunoglobulins; it also includes synthetic and genetically engineered variants of these immunoglobulins.

### Sample Processing

In some embodiments, the method further comprises the step of delivering the sample to a laboratory. For example, the entire device is frozen and shipped it to analysis location. At the analysis location sample may be extracted to a standard laboratory vial. An internal container in the sampling device may be punctured by two needles, one which allows air to be blown in and one which allows sample to be vented out into a standard laboratory vial. The sample may be blown out with air or a liquid diluent. A sample contained in an absorptive media may be transferred to a diluent and washed out of the absorptive media. Further spinning down the sample containing diluent may concentrate the protein component for further analysis.

In some embodiments, the laboratory patient information is masked to protect the identity of the subject when the sample is transferred to and from the laboratory. In further embodiments, the laboratory has means to identify multiple samples coming from a unique individual without identifying the individual. For example, the means to identify multiple samples coming from a unique individual can be a biochemical signature in the samples. More specifically, the means to identify a unique individual can include DNA sequencing and a lossy hash (an encryption technique) including a secret key. Alternately, the means to identify the multiple samples coming from a unique individual include a one-way encoding scheme transmitted from the doctor. In some embodiments, the laboratory uses multiple identified samples to construct an archetypal biochemical history of a disease process.

The sample can include additional features to facilitate analysis. In some embodiments, each device kit is labeled with a unique identifier (UID). This identifier is reproduced on multiple points within the kit. It is available externally, outside sterile containment, such that a health care provider (HCP) may enter the unique identifier into the patient record before device use. This UID label is preferably provided in all of machine readable, human readable, and adhesively affixable formats for easy transfer to medical records of various formats and interfaces. The UID is reproduced at multiple points where it may be useful for the tracking of the sample for later analysis, or for a second chance at entry in the patient record in case this critical step was skipped prior to the disposal of sterile packaging. A code of sufficient length and randomness is used such that incorrect data associated by a simple transcription error is extremely unlikely. The code length may also be defined such that one or more transcription errors may be tolerated while still providing a high confidence of a patient match and not substantially reducing security of the code.

The vitreous sampling device kit can include materials for shipping collected samples to an offsite laboratory for analysis. This includes a secondary container containing absorbent material, meeting requirements for shipping potentially infectious biosamples, into which the primary sample container should be placed prior to refrigeration. The label of the secondary container includes the UID as well as a simple form which can optionally be completed by an HCP.

The sample can be collected with other samples and shipped at least weekly to an offsite laboratory. A service for shipping the samples may regularly deliver a container, including refrigeration or a cryogenic such as wet or dry ice, into which the samples may be deposited and immediately returned to the shipping service. The container may serve a dual purpose to deliver unused devices to the practice.

At the laboratory the samples are received, cataloged, and queued for short term testing. A few microliters of each sample are transferred to a multiplexed array designed to identify the concentration of protein drug targets currently on the market with a highly sensitive antigen-antibody detection system. The remainder of each sample is frozen for later use. The results from the protein drug target test are immediately posted to a server for retrieval.

The vitreous sample may be pretreated as necessary by dilution in an appropriate buffer solution, concentrated if desired, or fractionated by any number of methods including but not limited to ultracentrifugation, fractionation by fast performance liquid chromatography (FPLC) or HPLC, or precipitation of proteins with dextran sulfate or other methods. Any of a number of standard aqueous buffer solutions at physiological pH, such as phosphate, Tris, or the like, can be used.

When the patient next arrives at the doctor's office at the typical interval after the first treatment (four weeks for typical antiVEGF treatment), an HCP opens the patient's chart and uses the UID to retrieve test results from the analysis system server. The interface to the server may be provided by a web page, local app, or accessed by the local or distributed patient medical record system via an API. In any case the access does not require transfer of the patient's identification. The results contain the patient's protein concentrations for several available drug targets as well as reference values showing population distributions for comparable patient populations. The results may be in the form of numeric values and reference intervals in a standard format familiar to an electronic medical record or may be in the form of a standard printable format such as PDF or Tiff that may be easily imported to a Picture Archiving and Communications System (PACS) next to imaging data and functional data most commonly used by eye doctors for patient diagnosis and tracking.

The laboratory may perform research on the samples that is not directly returned to the HCP to guide an immediate health care decision. In this case the laboratory refers to the entity that initially received the sample and performed the short-term immediate test, or a partner who performs additional testing and/or analysis on the sample and data. The purpose of this research may be to develop further tests which could be performed on similar samples. For example, initial test results may only provide the contraction of known drug targets, referenced to the distribution of those drug target concentrations in an initial clinical trial of a small population. Ongoing research may widen the range of drug targets analyzed in support of newly available drugs. Ongoing research may support referencing the concentration of the drug targets with increased statistical power as the pool of sampled patients increases. Alternatively, the samples may be analyzed for the presence of molecules which may form the basis of new treatments.

The anonymization resulting from not submitting the patients personal identifying information makes working with the patients' data easier and reduces risks of information leaks for the patient, HCP, and laboratory. Despite this anonymization HCPs are provided with a research consent form that should be provided to patients. The HCP is responsible to mark only samples for research for which a signed consent form is on record. An interface is provided whereby an HCP may request that a list of UID's be pulled from the research pool.

Many types of research may benefit from identifying features about the patient that are not communicated explicitly attached to the sample. It is extremely useful to know if multiple samples come from the same individual, demographic information about the patient, etc. DNA analysis is an extremely powerful tool that may provide reportable risk factors that may influence treatment, and can be used to provide the data mentioned above, but may in the future be able to explicitly identify any individual or be able to be used to impersonate an individual. For privacy reasons it is preferable that genetic data, if used, is only stored briefly and used to answer very specific questions, and then deleted. For example, a patients' ethnicity, presence of specific markers with known or suspected association to disease condition, and cryptographic hash of observed DNA sequence (e.g., MD5 or SHA), can preserve a large amount of scientific value, including the ability identify a future sample as coming from the same individual, without maintaining the ability to use the preserved database information to identify the patient or a close relative. Note that it is important to transmit right and left eye data with the eye explicitly, as this information is not presently extractable from DNA. It is also extremely useful to know the disease status of the eye at the time of sampling, its treatment history, and other disease conditions a patient may have concurrently. These factors may be available by measuring biomarkers that are not explicitly related to treatable signaling molecules pathways. For example the presence of blood or blood fractions in the eye may provide a direct indication of neovascularization and leaking vessels. Markers such as HbA1c may provide information about diabetes status. The concentration of drug in the eye from a previous dose may indicate the immediate treatment history of the eye. The combination of the ability to identify an eye, its current condition, and treatment history enables a researcher to build archetypal biochemical histories, which could be used to probe questions about responders vs non-responders and longer term prediction of treatment success without explicitly receiving that information in a structured clinical trial or requiring the effort of data entry from an HCP. Isolating a group of samples with similarities, or ensuring sufficient diversity within a group strengthens the ability to pool samples and perform analysis techniques that require larger samples, or are prohibitively expensive to perform on individual samples. Pooled samples are often currently used for example to perform label free proteomic studies for target discovery with high performance liquid chromatography coupled with mass spectrometry. Such label free techniques are critical for the identification of proteins of interest that may be investigated in greater with antigen-antibody techniques. Linking samples also enables the deletion of all samples and data from an individual by referencing a single UID.

### Methods of Biomarker Detection

The levels of protein biomarkers may be determined by any of a variety of standard protein analytic methods known in the art. These methods include absorbance, protein assays (e.g., DC protein assay, non-interfering protein assay, BCA protein assay, and the Lowry assay), gel electrophoresis (e.g., SDS-PAGE gel purification), a protein immunoblot (e.g., western blot), chromatography (e.g., size exclusion chromatography, ion exchange chromatography, and affinity chromatography), precipitation, ultracentrifugation, an immunoassay, such as an enzyme-linked immunosorbent assays (ELISA), mass spectrometry, and other common techniques known to one of ordinary skill in the art. In some embodiments, the assay used allows one to simultaneously determine the levels of multiple biomarkers in a vitreous sample. For example, an immunoassay using fluorescent imaging may be used, where the antibodies used to detect multiple protein biomarkers that emit at different fluorescent wavelengths so that their fluorescent signals can be easily distinguished.

An immunoassay is an assay that uses a binding ligand (e.g., an antibody) to specifically bind an antigen (e.g., a biomarker). An immunoassay is characterized by the use of specific binding properties of a particular binding ligand to isolate, target, and/or quantify the antigen. While use of aptamers as the binding ligand is not the use of a component of the immune system, assays using aptamers are nonetheless referred to herein as “immunoassays.” Specific binding to a binding ligand (e.g., antibody) under such conditions may require an antibody that is selected for its specificity for a particular protein. For example, polyclonal or monoclonal antibodies raised to a biomarker from specific species such as rat, mouse, or human can be selected that are specifically reactive with that biomarker and not with other proteins, except for polymorphic variants and alleles of the biomarker. This selection may be achieved by subtracting out antibodies that cross-react with the biomarker molecules from other species. Immunoassays can be run using a variety of different formats, including competitive homogenous immunoassays, heterogeneous immunoassays, one-site non-competitive immunoassays, and two-site non-competitive immunoassays.

In some embodiments, to detect microRNA or mRNA biomarkers, probes, primers, complementary polynucleotide sequences or polynucleotide sequences that hybridize to the microRNA products can be used. In some embodiments, reverse complementary polynucleotides serve as probes for microRNA markers.

In some embodiments, the biomarker is detected using a polynucleotide probe. The term “probe” as used herein refers to a polynucleotide sequence that will hybridize to a complementary target sequence. In one example, the probe hybridizes to a microRNA sequence. The probes provided herein have nucleotide sequences that have 90% sequence identity to polynucleotide sequences that are the complement of microRNA or mRNA biomarkers.

Quantitative PCR is another method suitable for detecting polynucleotide biomarkers, and is a method using a primer set capable of amplifying the sequence of the microRNA or mRNA and can measure the expression level of the present microRNA or mRNA; conventional quantitative PCR methods such as an agarose electrophoresis method, an SYBR green method, and a fluorescent probe method may be used. However, the fluorescent probe method is most preferable in terms of the accuracy and reliability of quantitative determination.

In some embodiments, the method includes one or more additional steps in which the results of the biomarker analysis are computationally processed to be more understandable to a care decision maker. For example, in some embodiments, the proteins are summarized into a descriptive score by weighting individual input measurements according to an action profile of the first treatment. Methods of weighing drug action profiles in drug bioinformatic analysis are known to those skilled in the art, and are described, for example, by S. Vilar and G. Hripcsak (Brief Bioinform. 2017 July; 18(4): 670-681. A drug action profile includes the proteins and other biochemical factors affected by the drug, as well as drug pharmacokinetics. In some embodiments, the biomarkers are weighted according to the action profile of the second treatment.

In some embodiments, the biomarker analysis is carried out in a laboratory where the laboratory analyzes the biomarkers to identify the current disease state of the individual. In some embodiments, the biomarkers used to identify the current disease state are protein biomarkers. Identification of the disease state can also include information provided by the doctor and included with the sample provided to the laboratory.

The laboratory can perform additional research on the sample, producing results that are not returned to the subject or the care provider (e.g., doctor). For example, the research can be used to contribute to the selection of future tests application to similar samples. The analysis can also contribute to future drug target discovery or validation. This research can consist of the analysis on a pooled sample combined from multiple patients. In some embodiments, the additional research is performed using label-free analysis techniques.

### Biomarker Analysis and Reporting

The value of reporting the biomarker signature is greatly enhanced by placing this information in the context of a treatment decision. Note that while decisions regarding a change of treatment are typically made by a care provider such as a physician, heuristics are frequently used to assist in these decisions, and these heuristics may be extended in some cases to automatic decision logic. While staying safely short of providing automatic decision logic, the biomarker signature can be partially processed to assist in using the heuristics applied by the doctor to make a treatment decision. In some embodiments, the biomarker signature is compared to a population distribution; i.e., the concentration of the biomarker(s) is compared to the concentration of biomarker(s) in patient populations to assist in narrowing a diagnosis to a biochemically specific phenotype: Useful population groups include populations of normal individuals, in populations of individuals who have the same disease and have responded well to the treatment, in populations of individuals who have the same disease who have responded poorly to treatment, in populations of individuals who have the same disease and who have responded well to other treatments. Accordingly, in some embodiments the population distribution is divided into patients who are responders or non-responders to the first treatment. In a further embodiment, the population distribution is divided into patients who are exhibiting an inflammatory reaction vs. those who are not exhibiting an inflammatory reaction. The report may assist the physician by performing a correlation calculation between the patient's biomarker profile and archetypal profiles of various relevant populations. The doctor may then classify the patient according to their best fit to a particular patient population, and make a decision regarding a possible change of treatment accordingly.

The structure of the report can convey complex information in an ordered way to guide a particular clinical decision. For example, in the decision between a first treatment agent and a second treatment agent, the results for each protein within of a multiprotein analysis can be plotted on axes which center on an estimate of a population central tendency and are scaled by the distribution of the population. The sign of the concentration can be reversed to clarify the decision. For example, proteins with antiangiogenic character may be represented inversely to proteins with angiogenic character. On an orthogonal axis the proteins may be ordered in relevance to a particular clinical decision. Proteins that have the highest statistical relevance favoring the success of treatment 1 may be plotted on the farthest left, while those proteins that have the highest statistical relevance favoring success of treatment 2 may be plotted on the farthest right. A protein which contraindicates treatment may be plotted conversely, i.e., a protein which militates against treatment 1 may be placed to the far right. Proteins which do not have a known statistical relevance to treatment success may be ordered by their action spectrum, such that proteins that are more certainly changed in a negative direction by treatment 1 than by treatment 2 are plotted to the left. Measured proteins, which may be generally important but do not strongly influence the decision between treatment 1 and treatment 2 may be plotted neutrally in the center of the plot. In this way, without fully understanding every reported value, a doctor may correctly interpret a plot favoring treatment 1 by observing that, in general, the plot has higher values on the left. In order to assist a decision based on action spectrum, a report may group elements which have similar reported biological functions, and may order them with response to the reported strength or certainty of those actions. This may be particularly useful when a doctor is weighing beneficial effects of a treatment vs unintended side effects. For example, a doctor may wish to attenuate a cluster of proangiogenic proteins while keeping an eye on a group of proinflammatory proteins. In a decision between maintaining a current treatment interval and changing to a longer or shorter treatment interval it may be useful to compare current patient values to threshold values which represent likely clinical outcomes as well as previous patient values considering the treatment interval before each of those previous values.

Summary values can be used an important tool to manage the complexity of the biomarker report. Weighted sums can summarize many factors into a single number. For example, if the relative contributions of many proangiogenic and antiangiogenic factors can be estimated, they may be summarized by a single number which generally indicates the likelihood of pathological angiogenesis. The results of the biomarker signature analysis can also include ratios that are included in the reporting of the biomarker signature. These are often useful when it is desirable to understand which of two factors dominates a particular situation. In some embodiments the ratio is a ratio of proangiogenic factors to antiangiogenic factors. For example, the ratio can be the ratio of angiopoietin 2 to angiopoietin 1, and/or a ratio of VEGF-A165a:VEGF-A165b. Alternately, or in addition, the ratio can be the ratio between angiogenic and inflammatory factors.

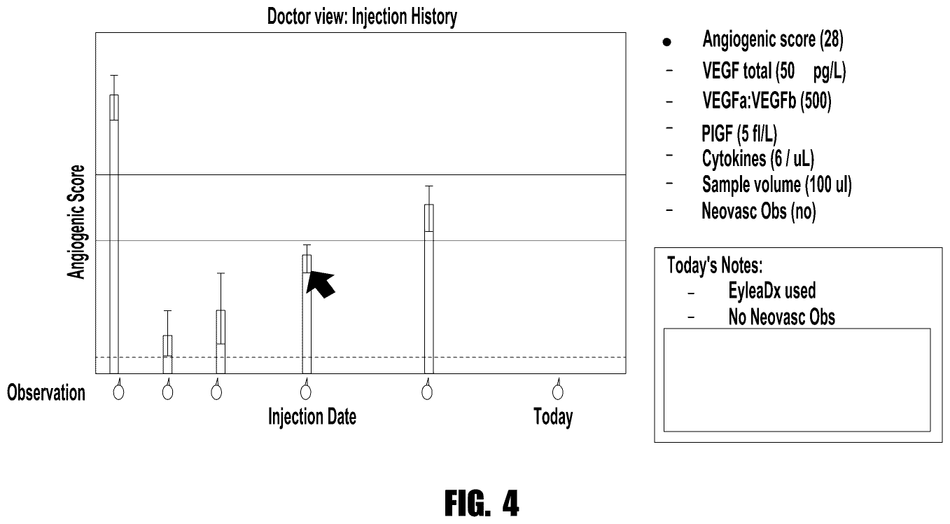

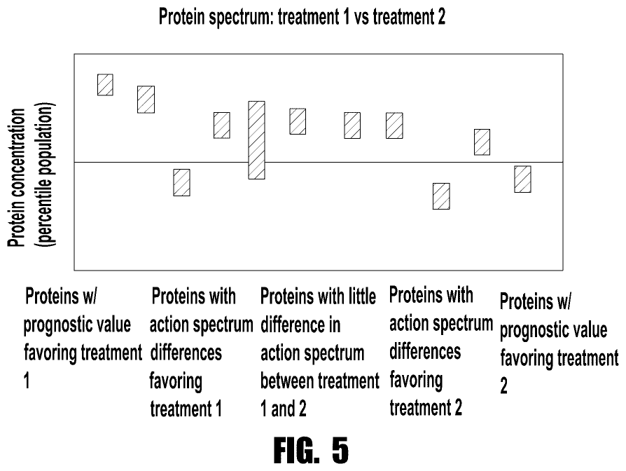

The method also includes reporting the biomarker signature of the sample. Reporting the biomarker signature includes communicating the results of the biomarker signature analysis to practitioner. For example, once the biomarker signature has been determined, it can be displayed in a variety of ways. For example, the levels can be displayed graphically on a display as numeric values or proportional bars (i.e., a bar graph) or any other display method known to those skilled in the art. The graphic display can provide a visual representation of the amount of the biomarker (e.g., polynucleotide or protein) in the vitreous sample being evaluated. In some embodiments, the analysis result is available in a standardized image form for storage in a picture archive and/or communications system. In some embodiments, the analysis result is available for retrieval by the care provider before the next scheduled visit of the subject. See for example FIG. 4 which provides a graph showing an embodiment of a doctor interface describing a few measured proteins and describing a history of treatments and results, and FIG. 5 which provides a bar graph indicating the concentration of multiple proteins in a sample.

Simultaneous to any immediate result which is passed back to the doctor (which can stimulate action by the patient and/or the doctor), a one-way masking system can be used to protect patient privacy and enable an offsite laboratory to perform forward looking research on the samples with a wide latitude and very low risk of divulging private patient data.

### Changing Treatment

The method also includes the step of changing the method of treatment to a second treatment if the biomarker signature indicates that use of the second treatment will have a preferable outcome. A preferable outcome, as defined herein, is one in which the patient recovers from disease, exhibits fewer symptoms of the disease, recovers more quickly, suffers fewer side effects, and/or requires less treatment. In some embodiments, a preferable outcome is indicated by a normalized protein signature. A normalized protein signature is one in which protein levels that are pathologically too high or low are changed to levels that correspond to those of a more healthy subject.

One can determine if a second type of treatment would be preferable by comparing the levels of biomarkers obtained with information that correlates these biomarker levels with a successful or unsuccessful outcome. In some embodiments, the biomarker is up-regulated in subjects that are responding poorly or successfully to the first treatment, while in other embodiments, the biomarker is down-regulated in subjects who are responding poorly or successfully to the first treatment. Other factors can be included in the determination, such as evaluation of the action profile, as described herein. For example, if the subject exhibits high levels of proteins that could be reduced or increased for a beneficial result using a different type of treatment, this can indicate that a more successful outcome could be obtained by switching to a second treatment.

In some embodiments, the decision on whether to change the type of treatment being received by the subject is also influenced by the patient's risk factors for the disease being treated. Risk factors for various eye diseases are known to those skilled in the art. For example, the risk factors for endophthalmitis include delayed diagnosis and treatment of microbial keratitis, the use of topical steroid, trauma, contact lens use and previous ocular surgical history.

Typically, the second treatment comprises the administration of a different agent. In some embodiments, the different agent is an agent taken from a different class of agents, as compared to the agent used in the first treatment. Alternately, or in addition, the second treatment comprises a different method of administration of the same agent—e.g., the use of a different method of administration, or different dosing schedule.

The concentration of biomarkers (e.g., signaling molecules) may be compared to the action spectrum of the potential drugs available. For example, in a typical patient the drug ranibizumab has been shown to attenuate free VEGF-A (which it binds directly) as well as has been shown to have attenuating effects on PIGF, IL-6, TGF-B, MCP-1, MIP-1a, MIP-1b, and ICAM-1. Similarly, alfibercept attenuates free VEGF-A, VEGF-B, and PIGF (which it binds directly) as well having attenuating effects on IL-1B, IL-IRA, IL-6, IL-10, IL-12p70, IL-12p40, Eotaxin-1, MIP-1a, MIP-1B, Flt-3L, and GRO, and amplifying effects on GM-CSF, and fraktaline. If the action spectrum of a potential drug (i.e., a potential second treatment) is more likely to affect the concentration of biomarkers in a way that will lead to a better outcome that the first treatment, the practitioner can change or recommend a change of treatment for the subject.

For example, where a subject is being treated for neovascular retinal disease (nvAMD, diabetic retinopathy) the doctor will usually choose a first line anti-VEGF agent (e.g., bevacizumab, ranibizumab, aflibercept) for the first IVI treatment. Analysis of the effect of the agent will involve determining the level of biomarkers in the vitreous fluid that stimulate or suppress neovascular growth, and those present which are correlated to stimulation or suppression of neovascular growth. However, analysis of the biomarker signature may indicated that high levels of IL-8 are present, and that a steroid should be used for the second treatment. (Kwon J., & Jee, D., PLOS One, 13(9), e0203408 (2018)). Steroid medications such as triamcinolone act to prevent transcription of inflammatory proteins at a genetic level. While this drug may still reduce levels of VEGF, its action is primarily to attenuate proinflammatory cytokines. (Torres-Costa et al., J Pharmacol Exp Ther., 373(3):445-452 (2020)).

The doctor may then choose the drug whose typical action spectrum has the highest correlation with the patient's abnormal presentation, in order to have the best chance of normalizing the protein signature of that patient. In some cases the differences between the drugs may be relatively subtle, in which case the decision may also strongly influenced by factors such as the cost or convenience of the drug. In other cases, the action of the drugs are strongly dissimilar. A basic classification of the disease state as primarily angiogenic, or primarily inflammatory may be useful to distinguish between an anti-VEGF vs a steroid drug.

Another situation involves treatment of age-related macular degeneration. If the biomarker signature indicates a high level of angiopoietin 2, this indicates that it might be effective to provide faricimab for the second treatment. If the biomarker signature indicated a high level of placental growth factor (PIGF). This indicates that it might be effective to provide aflibercept for the second treatment. Alternately, if high VEGFb or VEGFb: VEGFa is shown in the biomarker signature, it may be effective to provide pegaptanib for the second treatment. (Magnussen et al., Invest Ophthalmol Vis Sci., 51(8):4273-81 (2010)). In some embodiments, the biomarker signature may show high levels of one or more inflammatory cytokines, in which case it may be effective to provide a steroid for the second treatment. (Chalam et al., J Ophthalmol. 2014:502174 (2014)). In further embodiments, a biomarker signature exhibiting low VEGFa and low inflammatory cytokines indicates that photodynamic therapy may be effective for the second treatment.

The second treatment is selected from the group of set of drugs described herein as being suitable for the treatment of ophthalmic conditions. Examples of drugs suitable for treating ophthalmic conditions include anti-angiogenic agent suitable for ophthalmic conditions, antibiotics, and anti-inflammatory agents.

As described herein, the second treatment can also differ based on how a particular drug is being administered. Another decision made by the doctor is how frequently to deliver an IVI drug. In what is known as Treat and Extend protocol, the patient is given an IVI treatment at each doctor visit, and based on findings, the doctor sets the wait time before the next visit. Vitreous sample analysis may assist this decision by helping to stage the severity of disease, where more severe disease typically requires more frequent treatment. A particularly interesting case is that of treatment surprises. If a patient is routinely measured at a consistent interval, their angiogenic and inflammatory markers should be largely consistent from visit to visit. If those markers vary significantly from their typical values, the doctor should be wary of a treatment surprise. If proangiogenic and pro inflammatory markers appear higher than normal for a patient, the doctor may choose to shorten the time to the next visit and treatment. In all cases where a ‘doctor may choose’ automatic decision logic may be applied and the doctor may be given a strong recommendation to take a specific course of action. Accordingly, in various embodiments, the next scheduled visit is at least 1 day, at least one week, at least 2 weeks, or at least one month after the vitreous sample is obtained.

The complete disclosure of all patents, patent applications, and publications, and electronically available material cited herein are incorporated by reference. The foregoing detailed description and examples have been given for clarity of understanding only. No unnecessary limitations are to be understood therefrom. The invention is not limited to the exact details shown and described, for variations obvious to one skilled in the art will be included within the invention defined by the claims.

What is claimed is:

1. A method of guiding medical treatment comprising: analyzing a vitreous sample from a subject receiving an intravitreal injection first treatment, determining the biomarker signature of the sample, reporting the biomarker signature of the sample, and generating a recommendation to change the method of treatment to a second treatment if the biomarker signature indicates that use of the second treatment will have a preferable outcome.

2. The method of claim 1, wherein the method further comprises obtaining a vitreous sample from the subject.

3. The method of claim 1, further comprising administering the second method of treatment.

4. The method of claim 1, further comprising the step of delivering the sample to a laboratory.

5. The method of claim 1, wherein the biomarker signature of the sample is reported before the patient's next scheduled visit.

6. The method of claim 5, wherein the next scheduled visit is at least 2 weeks after the vitreous sample is obtained.

7. The method of claim 1, wherein the biomarker signature is a protein signature.

8. The method of claim 7, wherein the protein signature is determined using an immunoassay.

9. The method of claim 1, wherein the protein signature comprises a weighted sum of the concentrations of a plurality of proteins.

10. The method of claim 7, wherein the protein signature comprises angiogenic factors.

11. The method of claim 10, wherein the angiogenic factors comprise vascular endothelial growth factor (VEGF), placental growth factor (PIGF), and/or angiopoietin 2 (Ang2).

12. The method of claim 7, wherein the protein signature comprises inflammatory factors.

13. The method of claim 12, wherein the inflammatory factors comprise interleukin-8 (IL-8) and/or interleukin-6 (IL-6).

14. The method of claim 7, wherein the protein signature comprises proteins that are the known targets of ophthalmic drugs.

15. The method of claim 14, wherein the ophthalmic drugs are antibody-based drugs that specifically bind to the proteins.

16. The method of claim 7, wherein the proteins are weighted according to an action profile of the first treatment.

17. The method of claim 1, wherein the biomarkers are weighted according to an action profile of the second treatment.

18. The method of claim 1, wherein the biomarker signature is compared to a population distribution.

19. The method of claim 18 wherein the population distribution is divided into patients who are responders or non-responders to the first treatment.

20. The method of claim 1, wherein the first treatment comprises administration of an anti-angiogenic agent.

21. The method of claim 1, wherein the first treatment comprises administration of an antibiotic.

22. The method of claim 1, wherein the second treatment comprises administration of an anti-inflammatory agent.

23. The method of claim 1, wherein the biomarker signature includes bacterial antigens.

24. The method of claim 1, wherein the biomarker signature includes viral RNA.

25. The method of claim 1, wherein the biomarker signature is determined and reported a plurality of times.

26. The method of claim 1, wherein a preferable outcome is indicated by a normalized protein signature.

27. The method of claim 1, wherein the vitreous sample is obtained using a device that is configured to inject a volume into the eye having the same volume as the sample being obtained.

28. The method of claim 27, wherein the device is a double-barreled syringe comprising an injectant chamber and a sample chamber.
